(19) United States
(12) Patent Application Publication
(10) Pub. No.: US 2004/0253269 A1
(43) Pub. Date: Dec. 16, 2004

(54) Use of dictyotal extracts in the production of a topical composition

(75) Inventors: Gilles Gutierrez, Lyon (FR); Mostafa Serrar, Saint Bonnet De Mure (FR)

Correspondence Address: Charles A. Muserlian, c/o Muserlian, Lucas and Mercanti, 475 Park Avenue South, New York, NY, 10016, US

(73) Assignees: PARTICIPATIONS INDUSTRIELLES ET FINANCIERES S.A.; TEXINFINE S.A.

(21) Appl. No.: 10/813,388

(22) Filed: Mar. 30, 2004

Related U.S. Application Data
(62) Division of application No. 09/914,823, filed on Oct. 5, 2001.
Application No. 09/914,823 is a 371 of international application No. PCT/FR01/00067, filed on Jan. 10, 2001.

(30) Foreign Application Priority Data
Jan. 10, 2000 (FR) 00/00238

(51) Int. Cl. A61K 35/80

(52) U.S. Cl. 424/195.17

(57) ABSTRACT

The invention relates to the field of cosmetology, more specifically to novel topical compositions containing an alga extract.

The aim of the invention is to use an alga extract from the Dictyotal family in the production of compositions containing an alga extract as an active ingredient which results in the maturation of Keratinocytes and in the increase of desmosomial proteins for use by topical administration on the skin, mucuous membranes and teguments. The invention is used on parts of the body that are subjected to problems caused by ageing.

The present invention relates to the field of cosmetology and more especially to a new agent and new formulations intended for cosmetic purposes.

More especially it has as its aim new topical compositions containing an extract of algae, which have the property of activating the maturation of Keratinocytes.

Its specific aim is the use of a preparation based on extracts of an alga from the Dictyotales family with a view to producing a topical composition, intended for cosmetic purposes, bringing about the maturation of Keratinocytes with amplification of the synthesis of the cytokeratins, particularly cytokeratins CK1 and CK10, and the increase of desmosomial proteins to contribute to the consolidation of the stratified structure of the epidermis.

This extract of alga neither increases proliferation nor entails excessive ageing of the cells, and does not act on the cellular metabolism of the cells of human skin.

The epidermis which represents the surface section of the skin is formed from a succession of several layers of keratinocytes that are differentiated to a greater or lesser extent.

Thus, at the level of the basal layer, keratinocytes are found, which are small in size but have a great capacity for proliferation. Above this layer, there are several suprabasal layers made up of keratinocytes that are matured or developed, then differentiated as a function of their selective migration from the basal layer.

This stratified structure of the suprabasal layers of the epidermis can be modulated by several factors and particularly by soluble or ionic calcium—as opposed to fixed calcium: in the presence of calcium in a sufficient quantity, a stimulation of the synthesis of the cytoskeleton is observed and particularly of the cytokeratins, and of the assembling of cells by desmosomes, junction organisms made up of transcellular proteins necessary for better communication between the cells. These desmosomes play an important part in the organization of the cytoskeleton by making use of the anchorage sites for attaching cytokeratins.

It has also been demonstrated by several authors that the desmosomes were indispensable for the process of stratification by favouring the selective migration of cells in their final phase of differentiation, starting from the basal layer towards the suprabasal layers.

At the level of the cytoskeleton, an increase in the expression of cytokeratins is observed, particularly cytokeratins 1 and 10 which are markers of terminal differentiation under the effect of ionic calcium.

The extracts from “peacock's tail” weed, algae from the family of pheophyceae, have the property of activating the maturation of the keratinocytes.

The maturation of keratinocytes is observed in different situations: it is an essential stage in the evolution of keratinocytes, the epithelialization of the epidermic cells. It can be slowed down during certain illnesses such as psoriasis, or activated to an excessive degree in some other pathological conditions. Generally speaking, the maturation of keratinocytes is associated with an improvement in the mechanical properties of the skin. This improvement is linked to the very nature of the evolution of keratinocytes.

The maturation of keratinocytes results at cellular level in an increase in the proteins of the cytoskeleton involved in the maturation process, principally the cytokeratins 1 and 10, and in lesser quantities, the cytokeratins 5 and 13.

The maturation of keratinocytes results at tissue level in an improvement in the attachment and cohesion of the cells, linked with an increase in the expression of desmosomial proteins. The maturation of Keratinocytes is very active in the skin of young patients. Differentiation is expressed rapidly in the skin of old people. These characteristics are sought after by the cosmetics industry, which could be encouraged to place on the market compositions capable of endowing the skin with better qualities of resistance. The maturation of epithelial cells is moreover sought after when skin grafts are carried out, and in a general way during the culture of keratinocytes.

The maturation of keratinocytes is different from cellular ageing. It does not modify either proliferation or mitochondrial metabolism.

The substances generally used to bring about the maturation of keratinocytes accelerate differentiation and hence the ageing process. Initia division, then cause the collapse of proliferation. At the level of mitochondrial metabolism, they increase cellular respiration and then inhibit it.

At present, biologists do not have at their disposal substances likely to improve the differentiation of the keratinocytes without at term causing excessive ageing of the cells. Biological ageing shows itself by a decline and slowing down of general metabolism and particularly by a reduction in proliferation and synthesis potentials.

Now, surprisingly, it has just been discovered that certain ethanol or acetone extracts from a Dictyotale belonging to the class of pheophyceae, peacock's tail weed, indexed under the name Padina pavonica, have no effect on the proliferation of cells, that these extracts do not act on the pan Ras proto-oncogens. that these same extracts do not exacerbate or slow down mitochondrial metabolism (Test XTT or MTT), and that they nonetheless amplify the maturation of keratinocytes by a better expression of desmosomial proteins and cytokeratins, particularly CK1 and 10.

These extracts of Padina pavonica are defined by the process whereby they are obtained, in which the dried algae are steeped in ethanol or another organic solvent capable of evaporation. This raw extract is characterized by its biological activity on the keratinocytes after HPLC chromatography on C18 grafted silica. Eluted by a flow rate of one milliliter per minute with a mixture of methanol and water (90-10), the active fraction is situated between 9 and 12 minutes of retention. This raw extract is concentrated, then the raw extract phase is added depending on the level of activity found, or diluted in a vehicle chosen from an oil, a glycol, a wax, (jojoba oil, for example) and a paraffin or in a solid support such as cellulose, gelatine, silica or talc. This raw extract of Padina pavonica is thus incorporated in an excipient or a vehicle capable of forming compositions for cosmetic purposes. A titrated extract is thus obtained. To this end, the titrated extract of Padina pavonica is mixed or incorporated into one or more non-toxic inert excipients adapted for cosmetic use. In this connexion, mention may be made of diluting agents, thickening agents, dispersants, emulsifiers, surfactants, aromatizers, perfumes and/or other agents regulating the viscosity and/or preservatives.

The compositions according to the invention can take the form of creams, suspensions, W/O, O/W or Si/W emulsions, gels, pastes, ointments or powders.

Preferably, use will be made of supports intended to be diluted or incorporated into preparations such as creams or gels in which the extract of Padina pavonica is dispersed or diluted with its inert support after evaporation of the solvent and dilution with an inert support so as to produce the desired preparation, in an oily phase optionally containing non-ionic surfactants and/or silicones.

An aqueous phase possibly containing a gelling agent or a thickening agent or viscosity regulator is then added to the oily phase. After prolonged agitation, an emulsion is thus obtained, which is incorporated into a cosmetic base, fatty or non fatty, to produce a cream or a gel. The process is similar for preparations with a continuous oily phase.

It is also possible to incorporate the powder of a dry extract of Padina pavonica directly into a fatty base such as vaseline or lanolin, to form an ointment.

It is also possible to use the alcohol extract from Padina pavonica, to incorporate it into a polyethylene glycol solution containing a dispersant or surfactant, and add to it a silicon oil to produce a fluid emulsion which can, if desired, be coloured or perfumeed using a flower or natural plants essence or else a fragrant substance such as an ionone or lavendulol.

The cosmetic compositions contain from 0.1 g to 200 g extract of Padina per kg of the preparation, and in particular from 4 to 150 g per kg depending on the activity of the raw preparation and the qualities of the end product. It may be specified here that it is a matter of a concentration of the extract dosed in activity at 200,000 units of activity (UA) per litre.

The cosmetic compositions are intended for application to the body and particularly those parts of the body most exposed to the problems of ageing, such as the face, the arms, the upper body, neck and shoulders.

The compounds may be applied from one to four times per day, preferably from two to three times per day, over a period ranging from 10 to 60 days, preferably from 15 to 30 days. The active principle content ranges from 20 UA/kg to 80,000 UA/kg of final galenical preparation, and preferably from 2,000 to 40,000 UA/kg. The concentration is determined according to the activity of the final preparation and taking into account the fact that the active principle is made up of filtered raw extract.

Although the invention relates essentially to the use of topical preparations intended for cosmetic purposes, it is not impossible that a general use would lead to similar results.

A titre of 200,000 Units of activity/litre signifies that 20 μl of ethanol extract would reestablish at least 50% of the synthesis activity of glycosaminoglycanes, of 200,000 fibroblasts (cells extracted from explants of skin originating from healthy adult subjects, this variation being induced by the deleterious agent resulting from the addition of a solution of 20 IU/L of xanthine oxydase to 0.25 mMol of hypoxanthine). This system applies solely to the raw extract.

The invention extends further to the use of a preparation of extracts of seaweed of the Dictyotales family in culture media so as to increase the maturation of keratinocytes.

The biological activity of Dictyotales extracts was demonstrated by the tests below;

Padina Extract has no Effect on the Cellular Metabolism of the Cells of Human Skin.

Fibroblasts

XTT Test

TABLE 1

| | Control | 5 μl | 10 μl | 20 μl | 50 μl |
|---|---|---|---|---|---|
| Average | 0,269 | 0,250 | 0,272 | 0,241 | 0,261 |
| Standard deviation | 0,008 | 0,011 | 0,014 | 0,015 | 0,013 |

These figures are schematized by the histogram on the mitochondrial metabolism of the fibroblasts treated with an extract of Dictyotales, in FIG. 1.

Padina Extract has no Effect on the Cellular Proliferation of Cells of the Human Skin

Fibroblasts

Number of cells after 48 hours of culture

TABLE 2

| | Control | 5 μl | 10 μl | 20 μl | 50 μl |
|---|---|---|---|---|---|
| Average | 21,824 | 19,858 | 22,157 | 21,985 | 20,530 |
| Standard deviation | 704 | 654 | 641 | 384 | 518 |

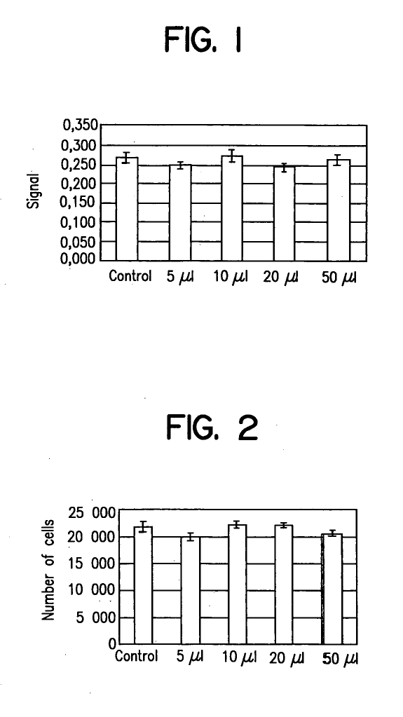

These figures are schematized by the histogram on the proliferation of fibroblasts treated with an extract of Dictyotales, in FIG. 2.

Keratinocytes

Number of cells after 48 hours of culture.

TABLE 3

| | Control | 5 μl | 10 μl | 20 μl | 50 μl |
|---|---|---|---|---|---|
| Average | 10 249 | 10 184 | 9 756 | 10 911 | 10 284 |
| Standard deviation | 655 | 507 | 587 | 584 | 445 |

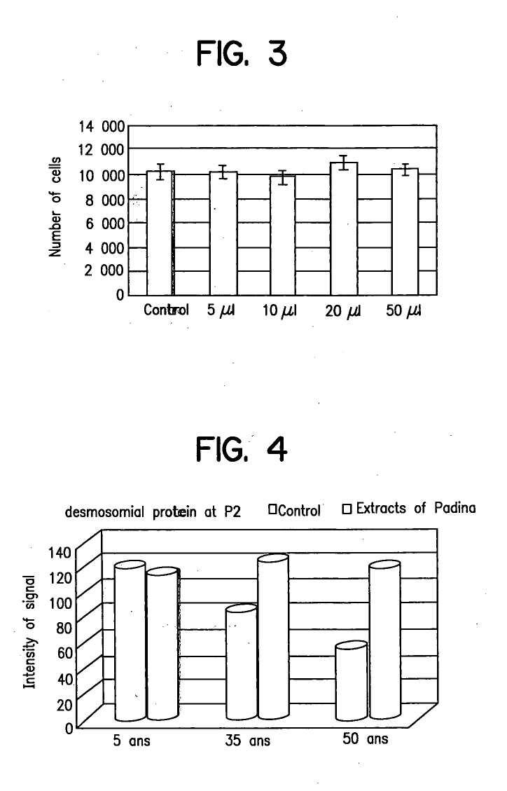

These figures are schematized by the histogram on the proliferation of keratinocytes treated with an extract of Dictyotales, in FIG. 3.

Padina Extract has an Effect on the Maturation of Keratinocytes Analysis of Cytokeratin 10

Principles

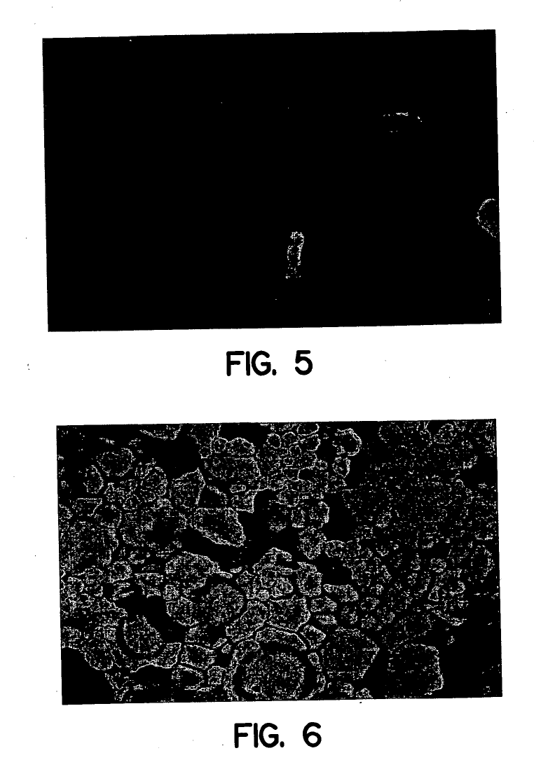

The antibodies directed against cytokeratin 10 are detected by means of a fluorescent conjugate. All the negative photographs are taken with the same enlargement and are reproduced as an appendix (FIGS. 5 and 6).

Results

Values

The photographs obtained according to the method described are digitized by a computer, which counts the pixels representing the points of fluorescence; the signal is calculated taking into account the total number of pixels on the photographs.

Results

Cytokeratin Signal

TABLE 4

| | 5 years | 35 years | 50 years |
|---|---|---|---|
| Control average | 15 | 10 | 5 |
| Standard deviation | 3 | 5 | 2 |
| Padina extract | 58 | 62 | 55 |
| Standard deviation | 3 | 4 | 8 |

The Extract of Padina has an Effect on the Maturation of Keratinocytes Analysis of Desmosomial Proteins

Principles

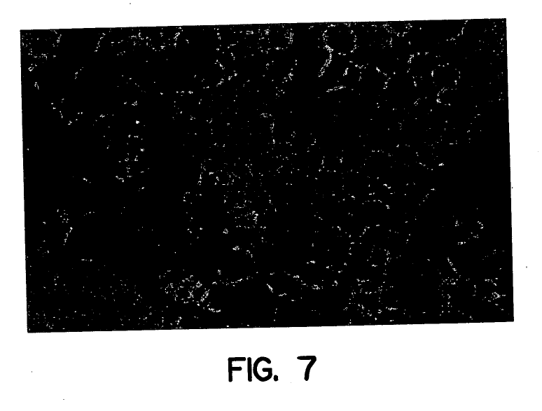

The antibodies directed against the desmosome proteins are detected by means of a fluorescent conjugate. All the photographs are taken with the same magnification (FIGS. 6 and 7).

Results

Values

The photographs obtained according to the method described are digitized by a computer which counts the pixels representing the points of fluorescence; the signal is obtained by calculating the total number of pixels on the plate.

Desmosomial Protein Signal

TABLE 5

| | 20 years | 40 years | 60 years |
|---|---|---|---|
| Control average | 120 | 105 | 55 |
| Standard deviation | 20 | 15 | 17 |
| Padina extract | 115 | 125 | 120 |
| Standard deviation | 15 | 10 | 20 |

These figures are schematized by the histogram on the desmosomial proteins at P2, in FIG. 4.

### EXAMPLE 1

Cream Based on Padina pavonica Extract Titrated at 200,000 Units/Litre

| | Ingredients | % |
|---|---|---|
| Phase A | Cetearyl alcohol and Ceteareth-20 | 3.5 |
| | Cetyl alcohol | 3.5 |
| | Octyl palmitate | 9 |
| | DL-□-Tocopherol Acetate | 0.1 |
| Phase B | De-ionized water | qsp 100 |
| Phase C | Casein —xanthane gum | 1 |
| Phase D | Preservative(s) | 1 |
| | Padina pavonica extract titrated at | 4 |
| | 200,000 units/liter | |
| | Xanthane gum | 0.3 |

Heat phase A to 75° C.

Heat phase B separately to 75° C.

Slowly add phase A to phase B under strong stirring.

Once this mixture is achieved, add phase C extemporaneously, constantly stirring vigorously to achieve homogenization.

Leave to cool until a temperature lower than 40° C. is obtained and add the elements of phase D separately while agitating less vigorously.

### EXAMPLE II

Gel Based on Padina pavonica Extract Titrated at 200,000 Units/Litre

| | Ingredients | % |
|---|---|---|
| Phase A | Glycerol | 4 |
| | Carbomere | 1 |
| Phase B | De-inonized water | qsp 100 |
| Phase C | PEG-78-glyceryl cocoate | 1 |
| | Padina pavonica extract titrated at | 2 |
| | 200,000 units/liter | |
| | Essential oil of bitter orange | 0.1 |
| Phase D | Preservative(s) | 0.7 |
| Phase E | NaOH diluted to 10% | qsp pH = 6 |

Add phase A to phase B.

Add phase C while agitating vigorously.

Once homogenization is complete, add D.

Stabilize the pH at 5-6 with dilute sodium hydroxide.

### EXAMPLE III

Milk Based on Padina pavonica Extract Titrated at 200,000 Units/Litre

| | Ingredients | % |
|---|---|---|
| Phase A | Cetearyl glucoside + cetearyl alcohol | 5 |
| | Sweet almond oil | 10 |
| | Jojoba oil | 5 |
| Phase B | De-ionized water | qsp 100 |
| | Citrate/citric buffer pH = 5.5 | |
| Phase C | Casein-xanthane gum | 1.5 |
| Phase D | Tocopherol | 0.1 |
| | Padina pavonica extract titrated at | 1 |
| | 200,000 U/liter | |
| | Preservative(s) | 0.7 |

Heat phase A to 75° C.

Heat phase B separately to 75%

Slowly add phase A to phase B under strong stirring.

Once this mixture is completed, add phase C extemporaneously while still stirring vigorously to ensure homogenization.

Leave to cool until a temperature below 40° C. is obtained and add the elements of phase D separately while stirring less vigorously.

### EXAMPLE IV

Cream Based on Padina pavonica Extract Titrated at 200,000 Units/Litre

| | Ingredients | % |
|---|---|---|
| Phase A | Paraffin oil | 10 |
| | Cetearyl alcohol | 1.5 |
| | Sorbitane stearate | 1.5 |
| | Polysorbate 60 | 2.5 |
| Phase B | De-ionized water | qsp 100 |
| Phase C | Extract of Padina pavonica titrated at | 10 |
| | 200,000 units/liter | |
| Phase D | Polyacrylamide and isoparaffin | 1.4 |
| | C13-C14 and Laureth-7 | |
| | Preservative | 0.7 |

Heat phase A to 75° C.

Heat phase B separately to 75° C.

Slowly add phase A to phase B agitating vigorously.

Leave to cool while agitating slightly until a temperature of 40° C. is obtained and add phase C while stirring vigorously.

When the mixture is quite homogeneous, add the elements of phase D separately.

1-11. (cancelled)

12. A method of allowing consolidation of stratified architecture of epidermis and insuring the improvement of the mechanical properties of the skin without inducing any proliferation of the cells, in a warm-blooded animal comprising applying on the parts of the body of a warm-blooded animal in need thereof susceptible to showing aging, an effective amount of a topical composition containing an amount of an extract of Padina pavonica effective to promote the maturation of Keratinocytes.

13. The method of claim 12 wherein the extract of Padina pavonica is provided by macerating the algae in the solvent to be evaporated.

14. The method of claim 12 wherein the crude extract resulting from the evaporation of the solvent, is titrated and incorporated into a formulation.

15. The method of claim 12 wherein the effective amount of the active extract of Padina pavonica ranges from 0.1 to 200 g per kg of the composition.

16. The method of claim 14 wherein the crude extract is used after titration of such or diluted in an inert cosmetic vehicle.

17. The method of claim 14 wherein the crude extract is incorporated in an excipient or a vehicle suitable for the realization of cosmetic compositions.

18. The method of claim 12 wherein the topical composition is in the form of creams, suspensions, emulsions of the W/O, O/W or Si/W types, of gels, of pastes, or pomades or powders.

19. The method of claim 12 wherein the concentration of extract of Padina pavonica in the cosmetic composition is such that the cosmetic composition contains from 0.1 to 200 g of Padina extract per kg of the composition.

20. The method of claim 12 in which a preparation of algae extract of the family of Dictyotales is used in a culture medium to increase the maturation of Keratinocytes.

21. The method of claim 12 wherein the titrate of 200,000 active units of the extract of Padina is defined in that 20 μl of crude extract is at least 50% of the synthetic activity of glycosaminologlycans of 200,000 fibroblasts.
